(19) United States
(12) Patent Application Publication
(10) Pub. No.: US 2023/0347048 A1
(43) Pub. Date: Nov. 2, 2023

(54) TREATMENT DEVICE OF CEREBROVASCULAR DISORDER

(71) Applicant: TERUMO KABUSHIKI KAISHA, Tokyo (JP)

(72) Inventors: Yu OSAWA, Ebina-city (JP); Takayuki UCHIDA, Hiratsuka-city (JP)

(73) Assignee: TERUMO KABUSHIKI KAISHA, Tokyo (JP)

(21) Appl. No.: 18/348,645

(22) Filed: Jul. 7, 2023

Related U.S. Application Data
(63) Continuation of application No. PCT/JP2022/001910, filed on Jan. 20, 2022.

(30) Foreign Application Priority Data
Feb. 26, 2021 (JP) 2021-030221

(51) Int. Cl. A61M 5/168

(52) U.S. Cl. CPC A61M 5/16877; A61M 2210/0693; A61M 2202/0464; A61M 2202/0476

(57) ABSTRACT

A treatment device of a cerebrovascular disorder capable of reliably controlling an oxygen concentration of a brain tissue so as not to be excessively high while efficiently supplying oxygen to the brain is provided. The treatment device of a cerebrovascular disorder includes: a main body including an injection unit that is to be inserted into a living body and injects a fluid; oxygen concentration measurement units disposed at two or more locations in the living body; and a flow rate adjustment unit that adjusts a flow rate of the fluid to be injected by the injection unit, in which the flow rate adjustment unit adjusts the flow rate of the fluid to be injected by the injection unit according to oxygen concentrations at two or more locations in the living body measured by the oxygen concentration measurement units.

### CROSS-REFERENCES TO RELATED APPLICATIONS

This application is a continuation of International Application No. PCT/JP2022/01910 filed on Jan. 20, 2022, which claims priority to Japanese Application No. 2021-030221 filed on Feb. 26, 2021, the entire content of both of which is incorporated herein by reference.

### TECHNOLOGICAL FIELD

The present disclosure generally relates to a treatment device of a cerebrovascular disorder, which is inserted from the spine and delivers a hyperoxygenated solution (including a high oxygen solution) to the brain.

### BACKGROUND DISCUSSION

In a treatment of cerebral infarction, treatment methods such as thrombolytic therapy and thrombus recovery therapy have been established. However, there are a lot of cases where these treatments are difficult due to a risk of bleeding and influence of an access failure.

As one of the treatments for cerebral infarction, it is conceivable to approach the brain with a hyperoxygenated solution and deliver oxygen from a source other than a blood vessel. As a treatment device for injecting a hyperoxygenated cerebrospinal fluid for treating cerebral infarction, is described, for example, in U.S. Pat. No. 4,686,085 B.

By continuing circulation of a hyperoxygenated solution at a relatively high flow rate to the brain, there is a risk of occurrence of a hyperoxia disorder, increase in an intracranial pressure, and the like. U.S. Pat. No. 4,686,085 B describes controlling a flow rate and an oxygen concentration of a solution on the basis of an oxygen concentration of a cerebrospinal fluid in the cisterna magna, but does not disclose a specific control method. In addition, although the oxygen concentration in the cisterna magna is measured, it is not clear whether or not a hyperoxia order, or the like, can be sufficiently prevented.

### SUMMARY

A treatment device for a cerebrovascular disorder is disclosed, which is capable of reliably controlling an oxygen concentration of a brain tissue so as not to be excessively high while efficiently supplying oxygen to the brain.

A treatment device of a cerebrovascular disorder according to the present disclosure includes: a main body including an injection unit that is to be inserted into a living body and injects a fluid; oxygen concentration measurement units disposed at two or more locations in the living body; and a flow rate adjustment unit configured to adjust a flow rate of the fluid to be injected by the injection unit. The flow rate adjustment unit are configured to adjust the flow rate of the fluid to be injected by the injection unit according to oxygen concentrations at the two or more locations in the living body measured by the oxygen concentration measurement units.

Further, a treatment device of a cerebrovascular disorder according to the present disclosure includes: a main body including an injection unit configured to be inserted into a living body and to inject a fluid; oxygen concentration measurement units configured to be disposed at two or more locations in the living body; and an oxygen concentration adjustment unit configured to adjust an oxygen concentration of the fluid to be injected by the injection unit. The oxygen concentration adjustment unit adjusts the oxygen concentration of the fluid to be injected by the injection unit according to oxygen concentrations at the two or more locations in the living body measured by the oxygen concentration measurement units.

Still further, a treatment device of a cerebrovascular disorder according to the present disclosure includes: a main body including an injection unit configured to be inserted into a living body and to inject a fluid and a discharge unit configured to discharge the fluid; a pressure measurement unit disposed in a flow path of the main body; and a flow rate adjustment unit configured to adjust a flow rate of the fluid to be injected by the injection unit or a flow rate of the fluid to be discharged by the discharge unit. The flow rate adjustment unit adjusts the flow rate of the fluid to be injected by the injection unit or the flow rate of the fluid to be discharged by the discharge unit according to a pressure in the living body measured by the pressure measurement unit.

Yet further, a treatment device of a cerebrovascular disorder according to the present disclosure includes: a main body including an injection unit configured to be inserted into a living body and to inject a fluid; an oxygen concentration measurement unit configured to be disposed in the living body; and a flow rate adjustment unit configured to adjust a flow rate of the fluid to be injected by the injection unit. The flow rate adjustment unit decreases the flow rate or stops supply of the fluid to be injected by the injection unit in a case where an oxygen concentration in the living body measured by the oxygen concentration measurement unit exceeds a predetermined upper limit value.

The treatment device of a cerebrovascular disorder configured as described above adjusts the flow rate or the oxygen concentration of the fluid to be injected by the injection unit on the basis of the oxygen concentrations at the two or more locations in the living body, and thus, can reliably control the oxygen concentration of the brain tissue not to be excessively high while efficiently supplying oxygen to the brain.

The flow rate adjustment unit may decrease the flow rate or stop supply of the fluid to be injected by the injection unit if any one of the oxygen concentrations at the two or more locations in the living body measured by the oxygen concentration measurement units exceeds the predetermined upper limit value. This makes it possible to provide a sufficient oxygen concentration to the fluid while preventing oxygen concentrations measured at a plurality of locations from exceeding the upper limit value, and thus, oxygen can be efficiently supplied to the brain.

After decreasing the flow rate or stopping supply of the fluid to be injected by the injection unit, the flow rate adjustment unit may adjust the flow rate of the fluid to be injected by the injection unit so that the oxygen concentrations at the two or more locations in the living body measured by the oxygen concentration measurement units are between the predetermined upper limit value and a predetermined lower limit value. As a result, the oxygen concentrations at a plurality of locations in the living body are controlled within a certain range, so that it is possible to reliably prevent the oxygen concentration of the brain tissue from becoming excessively high.

The oxygen concentration adjustment unit may decrease the oxygen concentration of the fluid to be injected by the injection unit if any one of the oxygen concentrations at the two or more locations in the living body measured by the oxygen concentration measurement units exceeds the predetermined upper limit value. As a result, it is possible to provide a sufficient oxygen concentration to the fluid while preventing oxygen concentrations measured at a plurality of locations from exceeding the upper limit value, so that oxygen can be efficiently supplied to the brain.

After decreasing the oxygen concentration of the fluid to be injected by the injection unit, the oxygen concentration adjustment unit may adjust the oxygen concentration of the fluid to be injected by the injection unit such that the oxygen concentrations at the two or more locations in the living body measured by the oxygen concentration measurement units are between the predetermined upper limit value and a predetermined lower limit value. As a result, oxygen concentrations at a plurality of locations in the living body can be controlled within a certain range, so that it is possible to reliably prevent the oxygen concentration of the fluid from becoming excessively high.

In addition, in the treatment device of a cerebrovascular disorder configured as described above, the flow rate of the fluid to be injected by the injection unit or the flow rate of the fluid to be discharged by the discharge unit is adjusted according to a pressure in the living body measured by the pressure measurement unit, so that oxygen can be efficiently supplied to the brain and reliably controlled without a risk of increasing an intracranial pressure.

### BRIEF DESCRIPTION OF THE DRAWINGS

FIG. 1 is a configuration diagram of a treatment device of a cerebrovascular disorder of the present embodiment.

FIG. 2 is a front view of a main body.

FIG. 3 is a flowchart of a treatment using the treatment device of a cerebrovascular disorder in a case where a flow rate of a fluid is adjusted.

FIG. 4 is a graph indicating a first pattern of transition of an oxygen concentration with respect to time in a treatment using the treatment device of a cerebrovascular disorder.

FIG. 5 is a graph indicating a second pattern of transition of an oxygen concentration with respect to time in a treatment using the treatment device of a cerebrovascular disorder.

FIG. 6 is a flowchart of a treatment using the treatment device of a cerebrovascular disorder in a case where an oxygen concentration of the fluid is adjusted.

### DETAILED DESCRIPTION

Set forth below with reference to the accompanying drawings is a detailed description of embodiments of a treatment device of a cerebrovascular disorder, which is inserted from the spine and delivers a hyperoxygenated solution (including a high oxygen solution) to the brain. Note that dimensional ratios in the drawings may be exaggerated and different from actual ratios for convenience of description. Furthermore, in the present specification, a side of a main body 10 to be inserted into a living body will be referred to as a “distal end” or a “distal end side”, and a hand side to be operated will be referred to as a “proximal end” or a “proximal end side”. A technical scope of the present disclosure is not limited to the embodiment of the present disclosure described below.

In a treatment device of a cerebrovascular disorder according to the embodiment of the present disclosure, the main body 10 is delivered to the vicinity of the brain, and a hyperoxygenated artificial cerebrospinal fluid (including an artificial cerebrospinal fluid) is injected into a subarachnoid space, and the cerebrospinal fluid is suctioned at a portion on the proximal end side from the subarachnoid space, thereby treating cerebral infarction.

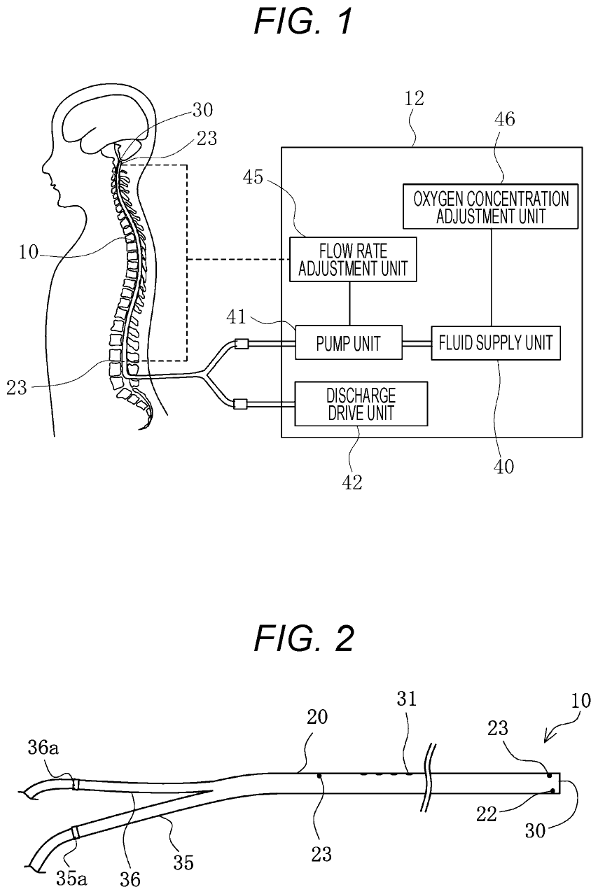

As illustrated in FIG. 1, the treatment device of a cerebrovascular disorder includes the main body 10 which has a tubular body to be inserted into a living body, and a control device 12 that adjusts a flow rate and an oxygen concentration of a fluid to be supplied to the main body 10. The main body 10 is inserted into the spinal canal from the lumbar spine of the human body, and a distal end portion is delivered to a position of the cisterna magna. An injection unit 30 into which a fluid flows is provided at the distal end portion of the main body 10. In addition, the main body 10 is provided with oxygen concentration measurement units 23 at two locations located in the living body. The oxygen concentration measurement units 23 are arranged on an outer surface at a distal end portion and the outer surface at a proximal portion of the main body 10. The oxygen concentration measurement unit 23 at the distal end portion is positioned in the vicinity of the cisterna magna and the oxygen concentration measurement unit 23 at the proximal portion is positioned in the vicinity of the lumbar spine in a state where the main body 10 is inserted into the living body (i.e., the subarachnoid space).

The control device 12 includes a fluid supply unit 40 that supplies oxygen to a fluid, a pump unit 41 that causes the fluid from the fluid supply unit 40 to flow to the main body 10, and a discharge drive unit 42 that discharges the fluid from the main body 10. An oxygen concentration adjustment unit 46 is connected to the fluid supply unit 40 and adjusts a concentration of oxygen to be supplied by the fluid supply unit 40. In addition, the control device 12 includes a flow rate adjustment unit 45 that is connected to the two oxygen concentration measurement units 23, receives information on the oxygen concentration of the fluid, controls the pump unit 41 on the basis of the information and adjusts the flow rate of the fluid to be supplied to the main body 10. The oxygen concentration measurement units 23 and the flow rate adjustment unit 45 are connected by a connection line provided along a longitudinal direction of the main body 10.

As illustrated in FIG. 2, the main body 10 includes a long tube main body (i.e., an elongated tubular body) 20. The tube main body 20 has a pressure measurement unit 22 on an outer surface at the distal end portion. Further, as described above, the tube main body 20 is provided with the oxygen concentration measurement units 23 at two locations. An injection unit 30 opened toward a distal end is formed at the distal end portion of the tube main body 20. A discharge unit 31 is formed in a middle portion of the tube main body 20. If the oxygen concentration measurement units 23 are disposed so as to protrude to the outside of the main body 10, it is concerned that the oxygen concentration measurement units 23 may be snagged by an insertion port when the main body is inserted into the lumbar spine. Thus, recesses may be provided in the main body 10, and the oxygen concentration measurement units may be disposed in the recesses such that it is prevented that the oxygen concentration measurement units snags the insertion port or others.

A proximal end portion of the tube main body 20 branches into two, an inlet tube 35 and an outlet tube 36. An inlet portion 35a is provided at a proximal end portion of the inlet tube 35. The pump unit 41 of the control device 12 is connected to the inlet portion 35a. An outlet portion 36a is provided at a proximal end portion of the outlet tube 36. The discharge drive unit 42 of the control device 12 is connected to the outlet portion 36a. As the inlet portion 35a and the outlet portion 36a, for example, a hub can be used. The inlet portion 35a and the injection unit 30 communicate with each other, and the outlet portion 36a and the discharge unit 31 communicate with each other by lumens along a longitudinal direction of the tube main body 20.

The tube main body 20 is preferably formed with a material having a certain degree of flexibility. Examples of such a material include polyolefin such as polyethylene, polypropylene, polybutene, an ethylene-propylene copolymer, an ethylene-vinyl acetate copolymer, an ionomer, or a mixture of two or more of the materials listed above, a soft polyvinyl chloride resin, polyamide, a polyamide elastomer, polyester, a polyester elastomer, polyurethane, a fluororesin such as polytetrafluoroethylene, silicone rubber, latex rubber, and the like.

Next, a treatment method using the main body 10 of the present embodiment will be described. In a case where a patient develops cerebral infarction, the main body 10 is percutaneously inserted into the living body and introduced into the subarachnoid space in the spinal canal through a gap between the lumbar spine or between the lumbar spine and the sacrum. Specifically, the main body 10 is introduced into the spinal canal using a space of L3-L4, L4-L5, or L5-S1 (i.e., space between lumbar vertebra L3 and lumbar vertebra L4, space between lumbar vertebra L4 and lumbar vertebra L5, or space between lumbar vertebra L5 and sacrum S1). However, the main body 10 may be introduced into the spinal canal from a position other than these positions.

The main body 10 introduced into the subarachnoid space in the spinal canal is inserted toward the brain. The main body 10 is inserted until the injection unit 30 at the distal end portion reaches the vicinity of the brain. A maximum insertion position of the main body 10 is desirably the cisterna magna.

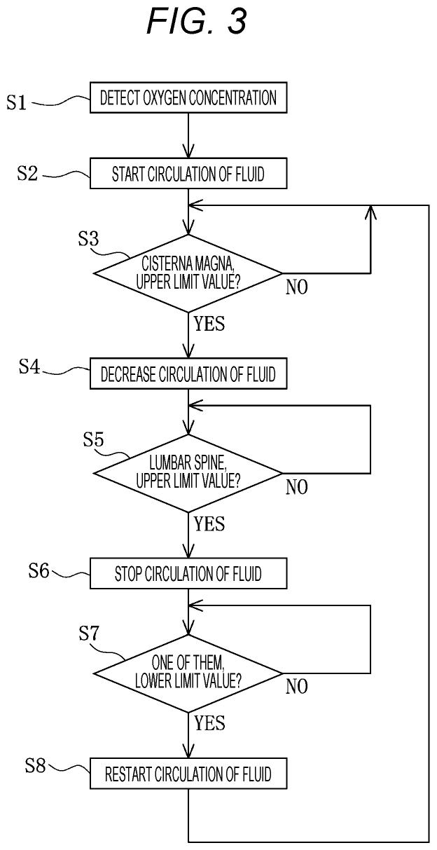

The process of adjusting a flow rate of the fluid will be described below. In the present example, the oxygen concentration adjustment unit 46 always keeps the oxygen concentration of the fluid to be supplied from the fluid supply unit 40 constant. When the main body 10 is inserted, as illustrated in FIG. 3, the flow rate adjustment unit 45 detects oxygen concentrations measured by the oxygen concentration measurement units 23 at two locations (S1). Then, the flow rate adjustment unit 45 starts driving of the pump unit 41 to start circulation of the fluid (S2). The fluid supplied from the pump unit 41 to the main body 10 is injected into the cerebrospinal fluid from the injection unit 30. When the fluid is supplied, the discharge drive unit 42 is also operated to suction the fluid (cerebrospinal fluid) in the spinal canal from the discharge unit 31. A suction amount of the fluid is preferably equal to an injection amount from the injection unit 30, which can help prevent an increase in intracranial pressure.

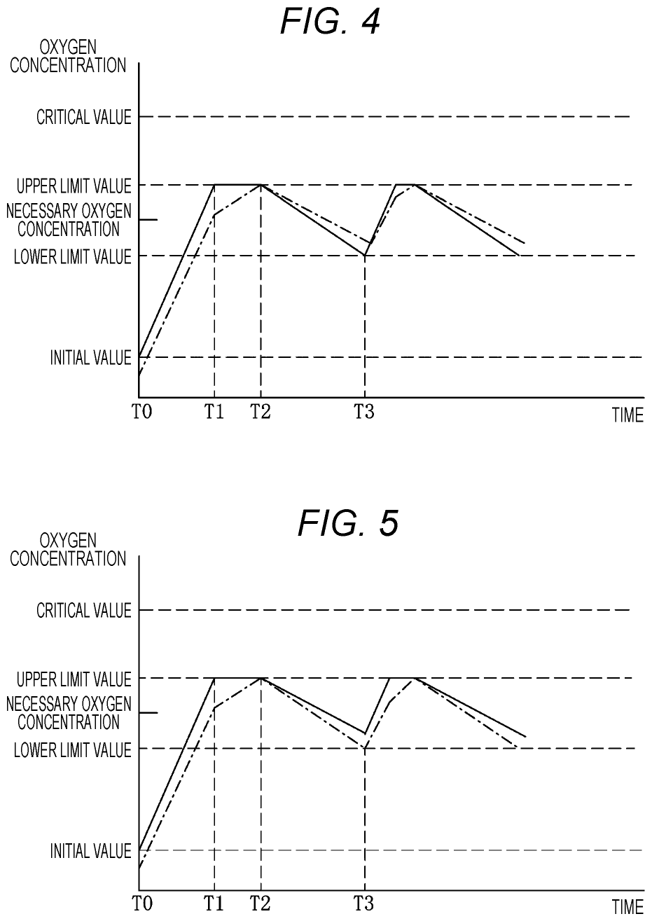

As illustrated in FIG. 4, an oxygen concentration at a location in the vicinity of the cisterna magna indicated by a solid line and an oxygen concentration at a location in the vicinity of the lumbar spine indicated by a one-dot chain line gradually increase from time TO at which supply of the fluid is started. The flow rate adjustment unit 45 detects oxygen concentrations measured by the oxygen concentration measurement units 23 at any time after S2 in this flow, and immediately stops supply of the fluid or decreases a flow rate of the fluid to be supplied in a case where the oxygen concentration reaches a critical value in any one of the oxygen concentration measurement units 23. In a case where the oxygen concentration in the vicinity of the cisterna magna does not increase even after supply of the fluid is started, or in a case where only the oxygen concentration in the vicinity of the cisterna magna increases and the oxygen concentration in the vicinity of the lumbar spine does not increase, the flow rate adjustment unit 45 determines that there is an abnormality in circulation, and the flow rate adjustment unit 45 stops the supply of the fluid.

As illustrated in FIG. 4, an upper limit value (i.e., an upper limit for the oxygen concentration) obtained by adding a certain value to a necessary oxygen concentration and a lower limit value (i.e., a lower limit for the oxygen concentration) obtained by subtracting a certain value from the necessary oxygen concentration are set in advance. The flow rate adjustment unit 45 performs control so that both the oxygen concentration in the vicinity of the cisterna magna and the oxygen concentration in the vicinity of the lumbar spine have values between the upper limit value and the lower limit value. The fluid for supplying oxygen is injected from the injection unit 30 to the vicinity of the cisterna magna, and thus, the oxygen concentration in the vicinity of the cisterna magna increases first. If circulation of the fluid is started in S2, the flow rate adjustment unit 45 determines whether or not the oxygen concentration in the vicinity of the cisterna magna has reached the upper limit value (S3). If the oxygen concentration in the vicinity of the cisterna magna reaches the upper limit value at time T1, the flow rate adjustment unit 45 decreases the flow rate of the fluid and decreases a circulation amount (S4). As a result, the oxygen concentration in the vicinity of the cisterna magna is controlled to be constant around the upper limit value. The flow rate of the fluid is controlled so that the oxygen concentration is maintained at about ±10% (i.e., plus or minus 10%) of the upper limit value. If the circulation amount (i.e., flow rate) of the fluid decreases, the rate of change in the oxygen concentration in the vicinity of the lumbar spine slows down and gradually increases toward the upper limit.

The flow rate adjustment unit 45 determines whether or not the oxygen concentration in the vicinity of the lumbar spine reaches the upper limit value (S5). If the oxygen concentration in the vicinity of the lumbar spine reaches the upper limit value at time T2, the flow rate adjustment unit 45 stops the circulation of the fluid (S6). As a result, both the oxygen concentration in the vicinity of the cisterna magna and the oxygen concentration in the vicinity of the lumbar spine decrease.

If the circulation of the fluid is stopped, the flow rate adjustment unit 45 determines whether or not any one of the oxygen concentration in the vicinity of the cisterna magna and the oxygen concentration in the vicinity of the lumbar spine has decreased to the lower limit value set in advance (S7). In FIG. 4, the oxygen concentration in the vicinity of the cisterna magna first decreases to the lower limit value at time T3. In this case, the flow rate adjustment unit 45 restarts circulation of the fluid at a timing of time T3 (S8). In FIG. 5, the oxygen concentration in the vicinity of the lumbar spine first decreases to the lower limit value at time T3. In this case, the flow rate adjustment unit 45 restarts circulation of the fluid at a timing of time T3 (S8). As a result, both of the oxygen concentrations start to increase again.

Thereafter, the processing returns to S3, and the same control is repeated. Thus, the oxygen concentration in the vicinity of the cisterna magna and the oxygen concentration in the vicinity of the lumbar spine can both be controlled to be between the upper limit value and the lower limit value. As a result, it is possible to help prevent the oxygen concentration from becoming excessively high while effectively supplying oxygen to the brain, and to reduce a risk of a high oxygen disorder, and the like.

During the circulation of the fluid, the intracranial pressure measured by the pressure measurement unit 22 is checked as needed. In a case where the intracranial pressure reaches or exceeds a certain value, the circulation of the fluid can be stopped.

The fluid collected by the discharge drive unit 42 is discarded as it is, but the collected fluid may be supplied to the brain together with the fluid from the fluid supply unit 40 after passing through a filter.

The circulation of the fluid can be performed, for example, for a certain period of time. If a certain period of time has elapsed, the circulation of the fluid is stopped, the main body 10 is removed from the living body, and the treatment is terminated.

In this example, the circulation of the fluid is stopped in S7, but the flow rate of the fluid may be made smaller than that when the circulation of the fluid decreases in S4, and the oxygen concentration of the fluid in the circulation may be controlled to decrease while maintaining the circulation of the fluid.

In addition, in this example, the oxygen concentration of the fluid is constant, and the oxygen concentration in the living body is controlled to be in a certain range by adjusting the flow rate of the fluid. However, the oxygen concentration in the living body may be controlled to be in a certain range by adjusting the oxygen concentration of the fluid while the flow rate of the fluid is made constant.

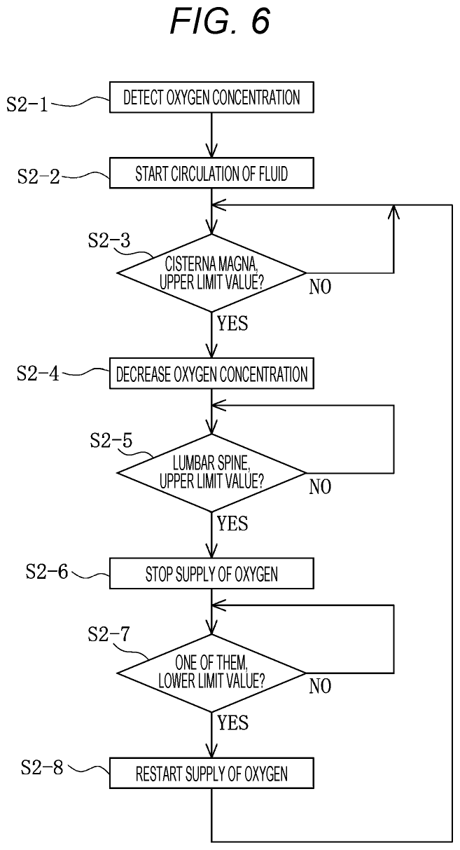

As illustrated in FIG. 6, S2-1 to S2-3 are similar to S1 to S3 in FIG. 3. In S2-4, the oxygen concentration adjustment unit 46 decreases the oxygen concentration of the fluid to be supplied from the fluid supply unit 40. As a result, as at time T1 to time T2 in FIG. 4, the oxygen concentration in the vicinity of the lumbar spine is increased while the oxygen concentration in the vicinity of the cisterna magna is kept constant around the upper limit value. If the oxygen concentration in the vicinity of the lumbar spine reaches the upper limit value (S2-5), the oxygen concentration adjustment unit 46 stops the supply of oxygen being supplied to the fluid (S2-6). Thereafter, if one of the oxygen concentration in the vicinity of the cisterna magna and the oxygen concentration in the vicinity of the lumbar spine decreases to the lower limit value (S2-7), the oxygen concentration adjustment unit 46 restarts supply of oxygen to the fluid (S2-8). As described above, the oxygen concentration in the vicinity of the cisterna magna and the oxygen concentration in the vicinity of the lumbar spine can be both controlled to be between the upper limit value and the lower limit value by adjusting the concentration of the oxygen to be supplied to the fluid, similarly to the case of adjusting the flow rate.

As described above, the treatment device of a cerebrovascular disorder according to the present embodiment includes the main body 10 including the injection unit 30 that is to be inserted into the living body and injects a fluid, the oxygen concentration measurement units 23 disposed at two or more locations in the living body, and the flow rate adjustment unit 45 that adjusts the flow rate of the fluid to be injected by the injection unit 30. The flow rate adjustment unit 45 adjusts the flow rate of the fluid to be injected by the injection unit 30 according to the oxygen concentrations at the two or more locations in the living body measured by the oxygen concentration measurement units 23. Alternatively, the treatment device of a cerebrovascular disorder according to the present embodiment includes: the main body 10 including the injection unit 30 that is to be inserted into the living body and injects a fluid; the oxygen concentration measurement units 23 provided at the two or more locations in the living body; and the oxygen concentration adjustment unit 46 that adjusts the oxygen concentration of the fluid to be injected by the injection unit 30. The oxygen concentration adjustment unit 46 adjusts the oxygen concentration of the fluid to be injected by the injection unit 30 according to the oxygen concentrations at the two or more locations in the living body measured by the oxygen concentration measurement units 23. The treatment device of a cerebrovascular disorder configured in this manner adjusts the flow rate or the oxygen concentration of the fluid to be injected by the injection unit 30 on the basis of the oxygen concentrations at the two or more locations in the living body, so that it is possible to reliably control the oxygen concentration of the fluid to be injected so as not to be excessively high while efficiently supplying oxygen to the brain. Here, the reason the oxygen concentration measurement units 23 are placed at two locations in the vicinity of the cisterna magna and in the vicinity of the lumbar spine will be described below. From the viewpoint of preventing a hyperoxia disorder, it is desirable to place the oxygen concentration measurement unit 23 in the brain, but it is difficult to place the oxygen concentration measurement unit 23 in the brain due to the structure of the human body, and thus, the oxygen concentration measurement unit 23 has to be placed at a location away from the brain. Thus, in the present embodiment, oxygen consumption and oxygen diffusion in the living body can be estimated by measuring the oxygen concentration in the vicinity of the lumbar spine away from the injection site in addition to the vicinity of the cisterna magna which is the injection site of the fluid.

In addition, the flow rate adjustment unit 45 may decrease the flow rate or stop the supply of the fluid to be injected by the injection unit 30 if any one of the oxygen concentrations at the two or more locations in the living body measured by the oxygen concentration measurement units 23 exceeds the predetermined upper limit value. This makes it possible to provide a sufficient oxygen concentration to the fluid while preventing oxygen concentrations measured at a plurality of locations from exceeding the upper limit value, and thus, oxygen can be efficiently supplied to the brain.

In addition, the flow rate adjustment unit 45 may adjust the flow rate of the fluid to be injected by the injection unit 30 such that the oxygen concentrations at two or more locations in the living body measured by the oxygen concentration measurement units 23 are between the predetermined upper limit value and the predetermined lower limit value after the flow rate of the fluid to be injected by the injection unit 30 is decreased or supply is stopped. As a result, the oxygen concentrations at a plurality of locations in the living body can be controlled within a certain range, so that it is possible to reliably prevent the oxygen concentration of the brain tissue from becoming excessively high.

In addition, the oxygen concentration adjustment unit 46 may decrease the oxygen concentration of the fluid to be injected by the injection unit 30 if any one of the oxygen concentrations at the two or more locations in the living body measured by the oxygen concentration measurement units 23 exceeds the predetermined upper limit value. As a result, it is possible to provide a sufficient oxygen concentration to the fluid while preventing oxygen concentrations measured at a plurality of locations from exceeding the upper limit value, so that oxygen can be efficiently supplied to the brain.

In addition, after the oxygen concentration of the fluid to be injected by the injection unit 30 is decreased, the oxygen concentration adjustment unit 46 may adjust the oxygen concentration of the fluid to be injected by the injection unit 30 such that the oxygen concentrations at the two or more locations in the living body measured by the oxygen concentration measurement units 23 are between the predetermined upper limit value and the predetermined lower limit value. As a result, the oxygen concentrations at a plurality of locations in the living body can be controlled within a certain range, so that it is possible to reliably prevent the oxygen concentration of the brain tissue from becoming excessively high.

In addition, the treatment device of a cerebrovascular disorder according to the present embodiment includes the main body 10 including the injection unit 30 that is to be inserted into the living body and injects the fluid and the discharge unit 31 that discharges the fluid, the pressure measurement unit 22 disposed in the flow path of the main body 10, and the flow rate adjustment unit 45 that adjusts the flow rate of the fluid to be injected by the injection unit 30 or the flow rate of the fluid to be discharged by the discharge unit 31. The flow rate adjustment unit 45 adjusts the flow rate of the fluid to be injected by the injection unit 30 or the flow rate of the fluid to be discharged by the discharge unit 31 according to the pressure in the living body measured by the pressure measurement unit 22. In the treatment device of a cerebrovascular disorder configured as described above, the flow rate of the fluid to be injected by the injection unit 30 or the flow rate of the fluid to be discharged by the discharge unit 31 is adjusted according to the pressure in the living body measured by the pressure measurement unit 22, whereby oxygen can be efficiently supplied to the brain and can be controlled without a risk of increasing the intracranial pressure.

The treatment device of a cerebrovascular disorder according to the present embodiment includes the main body 10 including the injection unit 30 that is to be inserted into the living body and injects the fluid, the oxygen concentration measurement unit 23 disposed in the living body, and the flow rate adjustment unit 45 that adjusts the flow rate of the fluid to be injected by the injection unit 30. In a case where the oxygen concentration in the living body measured by the oxygen concentration measurement unit 23 exceeds the predetermined upper limit value, the flow rate adjustment unit 45 decrease the flow rate or stops the supply of the fluid to be injected by the injection unit 30.

Note that the present disclosure is not limited to the above-described embodiment, and various modifications can be made by those skilled in the art within the technical idea of the present disclosure.

In the above-described embodiment, the oxygen concentration measurement units 23 are disposed at two locations in the vicinity of the cisterna magna and in the vicinity of the lumbar spine, but the oxygen concentration measurement units 23 may be disposed at three or more locations in the living body. In this case, the flow rate or the oxygen concentration of the fluid is adjusted such that the oxygen concentrations measured by all the oxygen concentration measurement units 23 are between the upper limit value and the lower limit value. In addition, the locations where the oxygen concentration measurement units 23 are disposed are not limited to the above-described locations, and the oxygen concentration measurement unit 23 can be disposed at a location, as necessary.

In the above-described embodiment, the main body 10 has a double lumen structure having a lumen communicating with the injection unit 30 and a lumen communicating with the discharge unit 31, but may have a single lumen or a multi-lumen structure.

In the above-described embodiment, the flow rate adjustment unit 45 adjusts the flow rate of the fluid on the basis of the oxygen concentrations measured by the oxygen concentration measurement units 23, but may adjust the flow rate of the fluid to be injected and the fluid to be discharged so that the pressure falls within a certain range (predetermined range) on the basis of the intracranial pressure measured by the pressure measurement unit 22. The pressure is constant in the subarachnoid space, and thus, control based on a pressure value measured at one location can be performed. The flow rate adjustment unit 45 adjusts a circulation amount of the fluid so that the pressure value measured by the pressure measurement unit 22 is between a preset upper limit value and a preset lower limit value. The location of the pressure measurement unit 22 is not limited to the location in the living body in the main body 10 and may be provided at any location in the flow path through which the fluid circulates.

The fluid supply unit 40 may perform temperature adjustment by cooling or heating the fluid to be supplied. The fluid supply unit 40 may have a valve such as a filter for passing the fluid or an air trap.

In the above-described embodiment, the main body 10 is inserted from the lumbar spine, but may be inserted from other positions as long as the main body 10 is a place where the subarachnoid space can be accessed, such as the thoracic spine, the cisterna magna, and the lateral ventricle.

In the above-described embodiment, the location of the injection unit 30 in the living body is in the vicinity of the cisterna magna, but may be other locations as long as the injection unit 30 is a place where the subarachnoid space can be accessed, such as the thoracic spine and the lateral ventricle.

A gas to be supplied to the fluid may be, for example, in addition to oxygen, another gas having a therapeutic effect on cerebral infarction, such as nitrogen monoxide, hydrogen, helium, or a mixed gas of two or more of nitrogen, monoxide, hydrogen and helium.

The fluid to be injected from the injection unit 30 may be other than an artificial cerebrospinal fluid. The fluid to be hyperoxygenated may be a liquid having high gas solubility, such as fluorocarbon, or an emulsion of fluorocarbon, or a solution that does not affect the living body, such as saline (or saline solution). In addition, the solution does not have to be hyperoxygenated and may be cooled or heated.

The treatment device of a cerebrovascular disorder can also be used for treatments other than cerebral infarction. For example, regarding cerebral diseases such as cerebral hemorrhage, subarachnoid hemorrhage, hydrocephalus, and Alzheimer's disease, and spinal cord ischemia, it is considered effective to inject a hyperoxygenated solution or forcibly circulate a cerebrospinal fluid, and the treatment device of a cerebrovascular disorder of the present embodiment can be used for these diseases.

The detailed description above describes embodiments of a treatment device of a cerebrovascular disorder, which is inserted from the spine and delivers a hyperoxygenated solution (including a high oxygen solution) to the brain. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A treatment device of a cerebrovascular disorder comprising:
a main body including an injection unit, the injection unit configured to be inserted into a living body and to inject a fluid into the living body;
oxygen concentration measurement units configured to be disposed at two or more locations in the living body; and
a flow rate adjustment unit configured to adjust a flow rate of the fluid to be injected by the injection unit.

2. The treatment device of the cerebrovascular disorder according to claim 1, wherein the flow rate adjustment unit is configured to adjust the flow rate of the fluid to be injected by the injection unit according to oxygen concentrations at the two or more locations in the living body measured by the oxygen concentration measurement units.

3. The treatment device of the cerebrovascular disorder according to claim 2, wherein the flow rate adjustment unit is configured to decrease the flow rate or to stop a supply of the fluid to be injected by the injection unit if one of the oxygen concentrations at the two or more locations in the living body measured by the oxygen concentration measurement units exceeds a predetermined upper limit value.

4. The treatment device of the cerebrovascular disorder according to claim 3, wherein the flow rate adjustment unit is configured to adjust the flow rate of the fluid to be injected by the injection unit such that the oxygen concentrations at the two or more locations in the living body measured by the oxygen concentration measurement units are between the predetermined upper limit value and a predetermined lower limit value after the flow rate of the fluid to be injected by the injection unit is decreased or the supply is stopped.

5. The treatment device of the cerebrovascular disorder according to claim 1, wherein the oxygen concentration adjustment unit is configured to adjust the oxygen concentration at the fluid to be injected by the injection unit according to oxygen concentrations at the two or more locations in the living body measured by the oxygen concentration measurement units.

6. The treatment device of the cerebrovascular disorder according to claim 5, wherein the oxygen concentration adjustment unit is configured to decrease the oxygen concentration of the fluid to be injected by the injection unit when one of the oxygen concentrations at the two or more locations in the living body measured by the oxygen concentration measurement units exceeds a predetermined upper limit value.

7. The treatment device of the cerebrovascular disorder according to claim 6, wherein after the oxygen concentration adjustment unit is configured to decrease the oxygen concentration of the fluid to be injected by the injection unit, the oxygen concentration adjustment unit configured to adjust the oxygen concentration of the fluid to be injected by the injection unit such that the oxygen concentrations at the two or more locations in the living body measured by the oxygen concentration measurement units are between the predetermined upper limit value and a predetermined lower limit value.

8. The treatment device of the cerebrovascular disorder according to claim 1, further comprising:
a pressure measurement unit disposed in a flow path of the main body, and wherein the flow rate adjustment unit is configured to adjust the flow rate of the fluid to be injected by the injection unit or the flow rate of the fluid to be discharged by a discharge unit according to a pressure in the living body measured by the pressure measurement unit.

9. The treatment device of the cerebrovascular disorder according to claim 1, wherein the injection unit of the main body is located at a distal end portion of the main body.

10. The treatment device of the cerebrovascular disorder according to claim 1, wherein the main body includes a discharge unit in a middle portion of the main body.

11. The treatment device of the cerebrovascular disorder according to claim 1, wherein a proximal end portion of the main body branches into an inlet tube and an outlet tube.

12. The treatment device of the cerebrovascular disorder according to claim 11, wherein the inlet tube includes an inlet portion at a proximal end portion of the inlet tube, and the outlet tube includes an outlet portion at a proximal end portion of the outlet tube.

13. The treatment device of the cerebrovascular disorder according to claim 12, further comprising:
a discharge unit configured to discharge the fluid, and wherein a flow rate of the fluid to be discharged by the discharge unit is adjusted by the flow rate adjustment unit.

14. The treatment device of the cerebrovascular disorder according to claim 13, wherein the inlet portion and the injection unit communicate with each other, and the outlet portion and the discharge unit communicate with each other by lumens extending along a longitudinal direction of the main body.

15. The treatment device of the cerebrovascular disorder according to claim 13, further comprising:
a discharge drive unit configured to suction a cerebrospinal fluid in a spinal canal of the living body from the discharge unit.

16. The treatment device of the cerebrovascular disorder according to claim 1, wherein the fluid is a hyperoxygenated artificial cerebrospinal fluid.

17. A treatment device of a cerebrovascular disorder comprising:
a main body including an injection unit, the injection unit is configured to be inserted into a living body and to inject a fluid and a discharge unit configured to discharge the fluid;
a pressure measurement unit disposed in a flow path of the main body;
a flow rate adjustment unit configured to adjust a flow rate of the fluid to be injected by the injection unit or a flow rate of the fluid to be discharged by the discharge unit; and
wherein the flow rate adjustment unit is configured to adjust the flow rate of the fluid to be injected by the injection unit or the flow rate of the fluid to be discharged by the discharge unit according to a pressure in the living body measured by the pressure measurement unit.

18. The treatment device of the cerebrovascular disorder according to claim 17, wherein flow adjustment unit is configured to maintain the pressure in the living body within a predetermined range as measured by the pressure measurement unit.

19. The treatment device of the cerebrovascular disorder according to claim 17, wherein the flow rate adjustment unit is configured to adjust a circulation amount of the fluid based on a pressure value measured by the pressure measurement unit, the pressure value being between a preset upper limit value and a preset lower limit value.

20. A treatment device of a cerebrovascular disorder comprising:
a main body including an injection unit, the injection unit being configured to be inserted into a living body and to inject a fluid into the living body;
an oxygen concentration measurement unit configured to be disposed in the living body;
a flow rate adjustment unit configured to adjust a flow rate of the fluid to be injected into the injection unit by the injection unit; and
wherein the flow rate adjustment unit is configured to decrease the flow rate or to stop a supply of the fluid to be injected by the injection unit in a case where an oxygen concentration in the living body measured by the oxygen concentration measurement unit exceeds a predetermined upper limit value.
